(19) United States
(12) Patent Application Publication
(10) Pub. No.: US 2004/0234475 A1
(43) Pub. Date: Nov. 25, 2004

(54) Oil-in-oil emulsions comprising a silicone, dispersions and use of said emulsions

(76) Inventors: Helene Lannibois-Drean, Charenton Le Pont (FR); Jean-Marc Ricca, Orchard Bel-Air (SG); Mathias Destarac, Paris (FR); Philippe Olier, Lyon (FR)

Correspondence Address: Jean Louis Seugnet, Rhodia Inc, Intellectual Property Department, 259 Prospect Plains Road CN 7500, Cranbury, NJ, 08512-7500, US

(21) Appl. No.: 10/481,168

(22) PCT Filed: Jun. 24, 2002
(86) PCT No.: PCT/FR02/02188

(30) Foreign Application Priority Data
Jun. 22, 2001 (FR) 01 08481

(51) Int. Cl. A61K 7/06; A61K 7/11

(52) U.S. Cl. 424/70.12

(57) ABSTRACT

The invention relates to emulsions comprising two non-aqueous, immiscible liquids, (L1) and (L2), whereby the liquid (L1), which is either the continuous or dispersed phase of the emulsion, is a silicone. The emulsion is further stabilized by at least one graft or block co-polymer of which one fraction is soluble in the dispersed phase the other in the continuous phase, the fraction soluble in the continuous phase being greater than the fraction soluble in the dispersed phase. The invention further relates to dispersions of said emulsions in an aqueous or organic phase and use of said emulsions and dispersions in the field of cosmetic and/or dermatological formulations

The present invention relates to emulsions comprising at least two mutually immiscible nonaqueous liquids, of which one is a silicone, and to their obtainment.

It likewise relates to dispersions of the emulsions having an external aqueous phase and to dispersions of the emulsions having an external organic phase.

The invention likewise pertains to the use of the emulsions and of the dispersions, particularly within the field of cosmetic and/or dermatological formulations.

More and more often consumers are being proposed a plurality of simultaneous and/or successive effects with a single formulation. Such performance is obtained by combining two or more active substances in the formulation.

This does not pose any major difficulty if the active substances are compatible with one another and with the medium of the formulation into which they are introduced.

When such cases do arise, the solution to these problems is to encapsulate the active substance so as to isolate it from the other active substance or from the medium into which it is introduced.

A first such solution has consisted in dispersing the active substance to be protected or the active substance for which a retarded effect is desired in the disperse phase of a simple direct emulsion. Although providing amelioration to the problem, this means of protection remains limited. The reason for this is that the oily barrier created between the active substance of the disperse phase and the medium of the formulation is not completely effective and diffusion of the active substance into the formulation is observed.

Another solution envisaged has been to prepare multiple emulsions: water/oil/water emulsions, for example. Thus descriptions have been given, inter alia, in French patent application FR 01 02397, filed 21 Feb. 2001, of cosmetic or dermatological formulations comprising at least two active substances in a multiple emulsion composed of an internal aqueous phase dispersed in an internal oily phase, this system being dispersed in an external aqueous phase, and the multiple emulsion being optionally mixed with a simple emulsion composed of an external oily phase dispersed in an external aqueous phase. According to that invention, each of the phases may comprise a hydrophobic or hydrophilic active substance, in accordance with the nature of the phase in question. Further, the inverse emulsion is stabilized by a nonionic surfactant and/or a nonionic amphiphilic polymer; the inverse emulsion is stabilized by a nonionic polyoxyalkylenated amphiphilic polymer and/or surfactant; the direct external emulsion is stabilized by a nonionic amphiphilic polymer and/or surfactant. This solution provides certain advantages when one of the active substances is hydrophilic.

One of the objects of the present invention is to provide an alternative solution for isolating at least one hydrophobic active substance from a formulation.

These objectives and others are achieved by the present invention, which accordingly first provides an emulsion comprising two nonaqueous liquids, L1 and L2, which are mutually immiscible, for which liquid L1, which represents the continuous phase of the emulsion or the disperse phase of the emulsion, is a silicone, the emulsion being stabilized by at least one comb or block copolymer of which one fraction is soluble in the disperse phase and the other in the continuous phase, the fraction soluble in the continuous phase being greater than the fraction soluble in the disperse phase.

The invention secondly provides a dispersion of the aforementioned emulsion in an external aqueous phase comprising a stabilizer comprising at least one comb or block copolymer of which one fraction is soluble in the disperse phase and the other in the continuous external aqueous phase, the fraction soluble in the continuous external oily phase being greater than the fraction soluble in the disperse phase.

The invention thirdly provides a dispersion of the aforementioned emulsion in a continuous external organic phase comprising at least one nonionic polyoxyalkylenated surfactant and/or at least one nonionic polyoxyalkylenated amphiphilic polymer.

The invention further provides a method of obtaining the emulsion according to the invention, in which, on the one hand, a first mixture comprising the disperse phase optionally comprising the active substance is prepared and, on the other hand, a second mixture comprising the continuous phase and the polymer is prepared, and then the first mixture is added to the second, with stirring.

The obtainment of the dispersion of the emulsion in a continuous external aqueous phase is likewise provided by the invention. It consists in preparing a mixture comprising the continuous external aqueous phase comprising the nonionic amphiphilic polymer and/or the surfactant, to which there is added the emulsion according to the invention, after which optionally, an aqueous thickener solution is added to the emulsion thus obtained.

As regards the obtainment of the dispersion of the emulsion in an external organic phase, it consists in preparing a mixture comprising the continuous external organic phase comprising the stabilizer, to which there is added the emulsion according to the invention.

The invention provides, finally, for the use of the emulsion, the dispersion in aqueous phase and the dispersion in organic phase in the field of the treatment of the skin and/or hair.

The emulsion and the dispersions according to the invention thus make it possible to isolate, to protect, one active substance from another active substance and/or from the formulation into which it is introduced, in order to avoid, for a sufficient period of time, any degradation which would result from the active substance coming into contact with another active substance/formulation. The invention represents an effective means of protecting hydrophobic active substances which are sensitive to hydrolysis and hydrophobic active substances which are sensitive to oxidation by atmospheric oxygen.

It should be noted—and this constitutes another advantage of the present invention—that it is possible to observe a retardation of the effect exerted by the active substance which is present in the disperse oily phase of the emulsion.

Moreover, the emulsion and the dispersions are a means of delivering an active substance. To cite just one example, the emulsions and dispersions according to the invention allow the combination, in a single formulation, of, on the one hand, a silicone, whose conditioning effect on the hair may be exerted at the time of application, and, on the other hand, a vegetable oil, whose nutrient/reparative effect on the hair will be obtained only after said oil has diffused through the silicone barrier deposited on the hair.

Finally, the sensorial effect resulting from the application of a formulation comprising the emulsion or one of the dispersions according to the invention is modified favorably, as is the texture of the formulation.

Still other advantages and characteristics of the invention will appear more clearly on reading the description, which now follows.

As indicated above, the emulsion according to the invention comprises two nonaqueous liquids, L1 and L2, which are mutually immiscible and of which one (L1) is a silicone.

By immiscible is meant any liquid which, at ambient temperature, has a solubility in another liquid that does not exceed 10% by weight.

Suitable liquids L1 or L2 may include compounds which are in liquid form at ambient temperature (20-30° C.) and/or at the temperature at which the emulsion is prepared.

As indicated before, the liquid L1 is selected from silicones.

Silicones which may be suitable for the implementation of the invention, alone or in mixtures, are silicones composed in whole or in part of units of formula:

R′₃₋ₐRₐSiO_{1/2} (unit M) and/or R₂SiO (unit D) in which formulae:

a is an integer from 0 to 3;

the radicals R are identical or different and represent:

a saturated or unsaturated aliphatic hydrocarbon group containing 1 to 10 carbon atoms;

an aromatic hydrocarbon group containing 6 to 13 carbon atoms;

a polar organic group bonded to the silicon by an Si—C or Si—O—C bond;

the radicals R′ are identical or different and represent:

a saturated or unsaturated aliphatic hydrocarbon group containing 1 to 10 carbon atoms;

an aromatic hydrocarbon group containing 6 to 13 carbon atoms;

an —OH function;

an amino- or amido-functional group containing 1 to 6 carbon atoms, bonded to the silicon by an Si—N bond.

Preferably at least 80% of the radicals R represent a methyl group.

These silicones may optionally comprise, preferably less than 5 mol % of, units of formulae T and/or Q:

RSiO_{3/2} (unit T) and/or SiO₂ (unit Q) in which formula R is as defined above.

By way of examples of aliphatic or aromatic hydrocarbon radicals R mention may be made of the following groups:

alkyl, preferably C₁-C₁₀ alkyl, optionally halogenated, such as methyl, ethyl, octyl or trifluoropropyl;

alkoxyalkylene, more particularly C₂-C₁₀, preferably C₂-C₆, such as —CH₂—CH₂—O—CH₃;

alkenyls, preferably C₂-C₁₀ alkenyl, such as vinyl, allyl, hexenyl, decenyl or decadienyl;

alkenyloxyalkylene such as —(CH₂)₃—O—CH₂—CH₂—O—CH═CH₂, or alkenyloxyalkoxyalkyl such as —(CH₂)₃—OCH₂—CH₂—O—CH═CH₂, in which the alkyl moieties are preferably C₁-C₁₀ and the alkenyl moieties are preferably C₂-C₁₀;

aryls, preferably C₆-C₁₃, such as phenyl.

By way of examples of polar organic groups R mention may be made of the following groups:

hydroxy-functional groups such as alkyl groups substituted by one or more hydroxyl or di(hydroxyalkyl)amino groups and optionally interrupted by one or more divalent hydroxyalkylamino groups. By alkyl is meant a hydrocarbon chain, preferably C₁-C₁₀, more preferably C₁-C₆; examples of these groups are —(CH₂)₃—OH; —(CH₂)₄N(CH₂CH₂OH)₂; —(CH₂)₃—N(CH₂CH₂OH)—CH₂—CH₂—N(CH₂CH₂OH)₂;

amino-functional groups such as alkyl substituted by one or more amino or aminoalkylamino groups, where alkyl is as defined above; examples thereof are —(CH₂)₃—NH₂; (CH₂)₃—NH—(CH₂)₂NH₂;

amido-functional groups such as alkyl substituted by one or more acylamino groups and optionally interrupted by one or more divalent alkyl-CO—N< groups, where alkyl is as defined above and acyl represents alkylcarbonyl, where one example is the group —(CH₂)₃—N(COCH₃)—(CH₂)₂NH(COCH₃);

carboxy-functional groups such as carboxyalkyl optionally interrupted by one or more oxygen or sulfur atoms, in which alkyl is as defined above; one example is the group —CH₂—CH₂—S—CH₂—COOH.

By way of examples of radicals R′ mention may be made of the following groups:

alkyl, preferably C₁-C₁₀ alkyl, optionally halogenated, such as methyl, ethyl, octyl or trifluoropropyl;

aryls, preferably C₆-C₁₃, such as phenyl;

amino-functional groups such as alkyl or aryl substituted by amino, alkyl being preferably C₁-C₆ and aryl denoting a cyclic aromatic hydrocarbon group, preferably C₆-C₁₃, such as phenyl; examples thereof are ethylamino and phenylamino;

amido-functional groups such as alkylcarbonylamino, where alkyl is preferably C₁-C₆; examples thereof are methylacetamido.

By way of specific examples of “units D” mention may be made of: (CH₃)₂SiO; CH₃(CH═CH₂)SiO; CH₃ (C₆H₅)SiO; (C₆H₅)₂SiO; CH₃ (CH₂—CH₂—CH₂OH)SiO.

By way of specific examples of “units M” mention may be made of: (CH₃)₃SiO_{1/2}; (CH₃)₂(OH)SiO_{1/2}; (CH₃)₂(CH═CH₂)SiO_{1/2}; (OCH₃)₃SiO_{1/2}; [O—C(CH₃)═CH₂]₃SiO_{1/2}; [ON═C(CH₃)]₃SiO_{1/2}; (NH—CH₃)₃SiO_{1/2}; (NH—CO—CH₃)₃SiO_{1/2}.

By way of specific examples of “units T” mention may be made of: CH₃SiO_{3/2}; (CH═CH₂)SiO_{3/2}.

When the silicones contain reactive and/or polar radicals R (such as OH, vinyl, allyl, hexenyl, aminoalkyls, etc.), these radicals do not generally represent more than 5% of the weight of the silicone, and preferably not more than 1% of the weight of the silicone.

As liquid L1 it is possible to use volatile oils such as hexamethyldisiloxane, octamethyl-disiloxane, decamethyltetrasiloxane, dodecamethyl-pentasiloxane, tetradecamethylhexasiloxane, hexa-decamethylhexasiloxane; heptamethyl-3[(trimethyl-silyl)oxy]trisiloxane, hexamethyl-3,3 bis[(trimethyl-silyl)oxy[trisiloxane; hexamethylcyclotrisiloxane, octamethylcyclotetrasiloxane, decamethylcyclopenta-siloxane, dodecamethylcyclohexasiloxane, pentamethyl-[(trimethylsilyl)oxy]cyclotrisiloxane.

It is likewise possible to employ nonvolatile silicones such as polydimethylsiloxane and α,ω-bis(hydroxy)polydimethylsiloxane gums and oils and also α,ω-bis(hydroxy)polydimethylsiloxane, polyphenylmethyl-siloxane and polydimethylsiloxane gums may likewise be used as liquid L1.

Preference is given more particularly to α,ω-bis(trimethyl)polydimethylsiloxane oils and α,ω-bis-(hydroxy)polydimethylsiloxane oils.

As representative silicones which are particularly suitable for the present invention mention may be made in particular of the silicones of polydimethylsiloxane(dimethicone) and diphenyl-dimethicone type.

As for the liquid L2 it is selected more particularly from:

organic waxes/fats/oils of animal or plant origin;

mineral waxes/oils;

products obtained from the alcoholysis of the aforementioned oils;

essential oils;

mono-, di- and tri-glycerides;

saturated or unsaturated fatty acids containing 10 to 40 carbon atoms; the esters of such acids with alcohol containing 1 to 6 carbon atoms;

saturated or unsaturated monoalcohols containing 2 to 40 carbon atoms;

polyols containing 2 to 10 carbon atoms;

silicones which have just been described; these compounds being used alone or in a mixture.

As organic waxes/fats/oils of animal origin mention may be made, inter alia, of cachalot oil, whale oil, seal oil, shark oil, cod liver oil, lard and mutton fat (tallows), perhydrosqualene and beeswax, alone or in a mixture.

By way of examples of organic waxes/fats/oils of plant origin, mention may be made, inter alia, of rapeseed oil, sunflower oil, peanut oil, olive oil, walnut oil, corn oil, soybean oil, avocado oil, linseed oil, hemp oil, grapeseed oil, copra oil, palm oil, cottonseed oil, babassu oil, jojoba oil, sesame oil, castor oil, macadamia oil, sweet almond oil, carnauba wax, karite butter, cocoa butter, and peanut butter, alone or in a mixture.

As regards the mineral waxes/oils, mention may be made, inter alia, of naphthenic oils, liquid paraffins (vaseline), isoparaffins, and paraffin waxes, alone or in a mixture.

The products obtained from the alcoholysis of the aforementioned oils may also be used.

Among essential oils mention may be made, without any intention of limitation thereto, of oils and/or essences of mint, spearmint, peppermint, menthol, vanilla, cinnamon, bay, aniseed, eucalyptus, thyme, sage, cedar leaf, nutmeg, citrus (lemon, lime, grapefruit, orange), and fruits (apple, pear, peach, cherry, prune, strawberry, raspberry, apricot, pineapple, grape, etc.), alone or in mixtures.

As regards the fatty acids, these acids, saturated or unsaturated, contain 10 to 40 carbon atoms, more particularly 18 to 40 carbon atoms, and may comprise one or more conjugated or nonconjugated ethylenic unsaturations. It should be noted that said acids may comprise one or more hydroxyl groups.

As examples of saturated fatty acids mention may be made of palmitic, stearic and behenic acids.

As examples of unsaturated fatty acids mention may be made of myristoleic, palmitoleic, oleic, erucic, linoleic, linolenic, arachidonic, and ricinoleic acids and also mixtures thereof.

As regards the fatty acid esters, mention may be made of the esters of the above-listed acids for which the part deriving from the alcohol contains 1 to 6 carbon atoms, such as methyl, ethyl, propyl and isopropyl esters, etc.

As an example of alcohols mention may be made of ethanol and of those alcohols corresponding to the aforementioned acids.

The internal organic phase (L2) may also be selected from mono-, di- and tri-glycerides.

Among the suitable polyols mention may be made with preference of glycerol.

The liquid L2 may also be a silicone or a mixture of two or more thereof, selected from those mentioned as being possible for use as liquid L1. It should be noted, however, that the silicones for L1 and L2 are selected such that they are mutually immiscible in the sense indicated before.

Finally, it is specified that the invention does not exclude the liquid L2 comprising an amount of water not exceeding the solubility limit of water in L2 (at a temperature of between 20 and 30° C.).

Depending on the selection of the various constituent elements, the liquid L1 (silicone) or else the liquid L2 may constitute the continuous phase of the emulsion. According to one preferred embodiment of the invention, the liquid L1 represents the continuous phase of the emulsion.

The proportion by weight of liquid L2 relative to liquid L1 is more particularly between 10/90 and 90/10, preferably between 30/70 and 50/50.

One of the features of the invention is that the oil-in-oil emulsion is stabilized by the presence of at least one comb or block copolymer one fraction of which is soluble in the disperse phase and the other in the continuous phase. Moreover, Bancroft's rule may be applied to this polymer, since the fraction soluble in the continuous phase is greater than the fraction soluble in the disperse phase.

More precisely, said copolymer is selected from those which are soluble in liquid L1 (silicone). More particularly, soluble copolymers are those which, when mixed with liquid L1, at a concentration of between 0.1 and 10% by weight of said liquid L1 and at 20° C., are in the form of a solution within all or part of the concentration range indicated.

Moreover, said copolymer is selected from those which, when mixed with liquid L2, at a concentration of between 0.1 and 10% by weight of said liquid L2 and at 20° C., are in the form of a dispersion within all or part of the concentration range indicated. In effect, the copolymer is partly soluble in said liquid.

Finally, said copolymer employed is one which, at a given concentration within the range indicated above and at 20° C., is present simultaneously in the form of a solution in L1 and in the form of a dispersion in L2.

The copolymer is advantageously not a crosslinked polymer.

Simply by way of indication it is specified that the copolymer has a number-average molar mass of less than or equal to 100 000 g/mol, more particularly between 1 000 and 50 000 g/mol. The number-average molar mass is given here with an absolute value, and may be advantageously determined by combining analysis by Malls (multiangle light scattering) with analysis by gel permeation chromatography. It should be specified that this method is particularly appropriate for copolymers of any mass. For copolymers of low mass, especially masses less than or equal to around 20 000 g/mol, it is also possible to use NMR, which also gives an absolute value for copolymer masses.

Advantageously, if each of the blocks of the copolymer represents one polymer (same size and composition as the blocks), then the monomers constituting each of the blocks will be selected such that each polymer is soluble, under the abovementioned temperature and concentration conditions, either in the liquid L1 (for the polymer deriving from the blocks soluble in L1) or in the liquid L2 (for the polymer deriving from the blocks soluble in L2).

More particularly, the fraction of the copolymer which is soluble in L1 derives from a polysiloxane, which may be selected from the silicones listed for L1 and L2.

The polysiloxane preferably carries reactive functional groups, such as the functions —OH and —NH₂, among others.

As regards the fraction soluble in L2, this fraction derives, preferably, from the polymerization of at least one monomer selected from the following monomers:

esters of linear, branched, cyclic or aromatic monocarboxylic or polycarboxylic acids comprising at least one ethylenic unsaturation;

α,β-ethylenically unsaturated nitriles, vinyl ethers, vinyl esters, vinylaromatic monomers, vinyl halides or vinylidene halides;

linear or branched, aromatic or nonaromatic hydrocarbon monomers comprising at least one ethylenic unsaturation;

alone or in mixtures, and also the macromonomers deriving from such monomers.

More specifically said monomers may be selected from:

the esters of (meth)acrylic acid with an alcohol containing 1 to 12 carbon atoms, such as methyl (meth)acrylate, ethyl(meth)acrylate, propyl (meth)acrylate, n-butyl(meth)acrylate, t-butyl (meth)acrylate, isobutyl(meth)acrylate, 2-ethylhexyl acrylate and hydroxyethyl (meth)acrylate;

vinyl acetate (which makes it possible to obtain partly or totally deacetylated polyvinyl alcohol), vinyl Versatate®, vinyl propionate, vinyl chloride, vinylidene chloride, methyl vinyl ether, ethyl vinyl ether, (meth)acrylonitrile, N-vinyl-pyrrolidone, vinylformamide, vinylacetamide and vinylamine,

(meth)acrylamide, N-alkyl(meth)acrylamide such as isopropyl acrylamide, and N-methylol (meth)acrylamide;

styrene, α-methylstyrene, vinyltoluene, butadiene, chloroprene and isoprene;

alone or in mixtures, and the macromonomers deriving from such monomers.

It will be recalled that the term “macromonomers” denotes a macromolecule carrying one or more polymerizable functions.

It should be noted that the fraction soluble in L2 of the copolymer employed as stabilizer of the emulsion according to the invention may be obtained from aforementioned monomers in combination with monomers of different chemical nature, such as, for example, ionic or nonionic hydrophilic monomers.

By way of example of ionic hydrophilic monomers, more particularly anionic or potentially anionic depending on the pH conditions, mention may be made in particular of:

linear, branched, cyclic or aromatic monocarboxylic or polycarboxylic acids, the N-substituted derivatives of such acids, and monoesters of polycarboxylic acids, comprising at least one ethylenic unsaturation;

linear, branched, cyclic or aromatic vinylcarboxylic acids;

amino acids comprising one or more ethylenic unsaturations;

alone or in mixtures, their sulfonic or phosphonic derivatives, the macromonomers deriving from such monomers, and the corresponding salts.

Possible examples of suitable monomers of this type, in acid form or in the alkali metal (sodium, potassium) or ammonium salt form, include the following:

(meth)acrylic acid, 2-propene-1-sulfonic acid, methallylsulfonic acid, styrenesulfonic acid, α-acrylamidopropanesulfonic acid, 2-sulfoethylene methacrylate, sulfopropyl(meth)acrylic acid, bis-sulfopropyl(meth)acrylic acid and the phosphate monoester of hydroxyethylmethacrylic acid;

vinylsulfonic acid, vinylbenzenesulfonic acid, vinylphosphonic acid and vinylbenzoic acid;

N-methacryloylalanine and N-acryloylhydroxy-glycine;

alone or in mixtures, and the macromonomers deriving from such monomers.

Nonionic hydrophilic monomers include the amides of linear, branched, cyclic or aromatic monocarboxylic or polycarboxylic acids containing at least one ethylenic unsaturation or derivatives such as (meth)acrylamide and N-methylol(meth)acrylamide; certain esters deriving from (meth)acrylic acid, such as, for example, 2-ethylhydroxyl(meth)acrylate; and vinyl esters which make it possible to obtain polyvinyl alcohol blocks after hydrolysis, such as vinyl acetate, vinyl Versatate®, and vinyl propionate.

If the copolymer forming part of the composition of the emulsion is obtained in part from hydrophilic monomers, it is specified that their amount is such that the copolymer meets the criteria set out previously, namely the fact that one fraction of said copolymer is soluble in L1, the other in L2, the fraction soluble in the liquid constituting the continuous phase of the emulsion being higher than that soluble in the disperse phase.

According to one preferred embodiment of the present invention the copolymer forming the part of the composition of the emulsion is a linear block polymer. Said copolymer preferably comprises at least three blocks.

The copolymers which can be used in the context of the present invention may be obtained advantageously by a free-radical route, preferably controlled.

With preference it is possible to obtain such polymers by employing a process for preparing, by thermal activation, hybrid silicone and organic copolymers comprising units (I):

RₓU_{y}SiO_{[4-(x+y)]/2} (I)

in which:

x is 0, 1, 2 or 3, y is 0, 1, 2 or 3, where 2≦(x+y)≦3 and y is other than 0 for at least one of the units of the hybrid copolymer,

the symbols R, which are identical or different, represent:

a linear or branched alkyl radical containing 1 to 8 carbon atoms, optionally substituted by at least one halogen, preferably fluorine, the alkyl radicals being preferably methyl, ethyl, propyl, octyl and 3,3,3-trifluoropropyl,

a cycloalkyl radical containing between 5 and 8 cyclical carbon atoms, which is optionally substituted,

an aryl radical containing between 6 and 12 carbon atoms, which may be substituted, preferably phenyl or dichlorophenyl,

an aralkyl moiety having an alkyl moiety containing between 5 and 14 carbon atoms and an aryl moiety containing between 6 and 12 carbon atoms, optionally substituted on the aryl moiety by halogens, alkyls and/or alkoxys containing 1 to 3 carbon atoms,

the symbols U which are identical or different, represent (II):

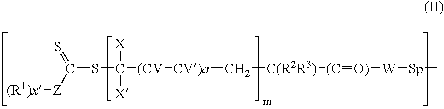

in which:

x′=1, 2, 3 or 4, depending on the valence of Z,

Z, which are identical or different, represent a carbon, sulfur, oxygen, phosphorus or nitrogen atom and/or a free valence,

R¹, which are identical or different, represent:

an optionally substituted alkyl, acyl, aryl, alkene or alkyne group (i),

a saturated or unsaturated carbon ring (ii), optionally substituted and/or aromatic, and/or

a saturated or unsaturated heterocycle (iii), optionally substituted,

it being possible for these radicals (i), (ii) and (iii) to be substituted advantageously by: substituted phenyl groups, substituted aromatic groups, or the following groups: alkoxycarbonyl, aryloxycarbonyl (—COOR⁵), carboxyl (—COOH), acyloxy (—O₂CR⁵), carbamoyl (—CONR⁵)₂, cyano (—CN), alkylcarbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups having a hydrophilic or ionic character such as the alkali metal salts of carboxylic acids, the alkali metal salts of sulfonic acid, polyalkylene oxide chains (PEO, PPO), cationic substituents (quaternary ammonium salts), with R⁵, which are identical or different, representing an alkyl or aryl group, and/or a polymer chain,

a group (iv) of formula —CₙF₍₂ₙ₊₁₎ where n is between 1 and 20,

a group (v) of the following formula:

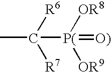

in which:

R⁶ and R⁷, which are identical or not, are selected from a halogen group, —NO₂, —SO₃R¹⁰, —NCO, —CN, —OR¹⁰, —SR^{10,} N(R¹⁰)₂, —COOR¹⁰, —O₂CR¹⁰, —CON(R¹⁰)₂, —NCO(R¹⁰)₂ and —CₙF₍₂ₙ₊₁₎, where n is between 1 and 20, and is preferably 1;

where R¹⁰ represents:

a hydrogen atom,

or an alkyl, alkenyl, alkynyl, cycloalkenyl, cycloalkynyl, alkaryl, aralkyl, heteroaryl or aryl radical optionally fused with an aromatic or nonaromatic heterocycle, it being possible for these radicals to be optionally substituted by:

one or more identical or different groups selected from halogen atoms, ═O, ═S, —OH, alkoxy, SH, thioalkoxy, NH₂, mono- or dialkylamino, CN, COOH, ester, amide, and CₙF₍₂ₙ₊₁₎ and/or optionally interrupted by one or more atoms selected from O, S, N and P;

or a heterocyclic group optionally substituted by one or more groups as defined above;

or R⁶ and R⁷, together with the carbon atom to which they are attached, form a group ═O, ═S, a hydrocarbon ring or a heterocycle;

and R⁸ and R⁹, which are identical or different, represent a group as defined above for R¹⁰, or together form a C₂-C₄ hydrocarbon chain, optionally interrupted by a heteroatom selected from O, S and N;

V and V′, which are identical or different, represent H, an alkyl group or a halogen,

X and X′, which are identical or different, represent H, a halogen or a group R⁴, OR⁴, O₂COR⁴, NHCOH, OH, NH₂, NHR⁴, N(R⁴)₂, (R⁴)₂N⁺O⁻, NHCOR⁴, CO₂H, CO₂R⁴, CN, CONH₂, CONHR⁴ or CONR⁴₂, in which R⁴ is selected from alkyl, aryl, aralkyl, alkaryl, alkene or organosilyl groups, which are optionally perfluorinated and optionally substituted by one or more carboxyl, epoxy, hydroxyl, alkoxy, amino, halogen or sulfonic groups,

R² and R³, which are identical or different, represent:

an optionally substituted alkyl, acyl, aryl, alkene or alkyne group (i),

a saturated or unsaturated carbon ring (ii), optionally substituted and/or aromatic,

a saturated or unsaturated heterocycle (iii), optionally substituted,

a hydrogen atom or the following groups: alkoxycarbonyl, aryloxycarbonyl (—COOR⁵), carboxyl (—COOH), acyloxy (—O₂CR⁵), carbamoyl (—CONR⁵₂), cyano (—CN), alkylcarbonyl, alkylarylcarbonyl, arylcarbonyl, arylalkyl-carbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups having a hydrophilic or ionic character such as the alkali metal salts of carboxylic acids, the alkali metal salts of sulfonic acid, polyalkylene oxide chains (PEO, PPO), and cationic substituents (quaternary ammonium salts), with R⁵, which are identical or different, representing an alkyl or aryl group and/or a polymer chain,

it being possible for the radicals (i), (ii) and (iii) to be substituted advantageously by: substituted phenyl groups, substituted aromatic groups, or the following groups: alkoxycarbonyl, aryloxycarbonyl (—COOR⁵), carboxyl (—COOH), acyloxy (—O₂CR⁵), carbamoyl (—CONR⁵₂), cyano (—CN), alkyl-carbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups having an ionic or hydrophilic character such as the alkali metal salts of carboxylic acids, the alkali metal salts of sulfonic acid, polyalkylene oxide chains (PEO, PPO), cationic substituents (quaternary ammonium salts), with R⁵, which are identical or different, representing an alkyl or aryl group and/or a polymer chain,

W, which are identical or different, represent a divalent radical selected from —O—, —NR⁴—, —NH—, and —S—,

Sp, which are identical or different, represent a coupling unit composed of a divalent organic radical of —(CH₂)_{x′″}-, in which x′″ is between 1 and 20, it being possible for this radical to be substituted and/or to contain at least one heteroatom,

a=0 or 1,

m≧1, and, when m>1, the repeating units of index m are identical or different,

in which process the following are contacted with one another:

at least one ethylenically unsaturated monomer of the formula (III):

CXX′(═CV—CV′)ₐ═CH₂

a precursor silicone compound comprising identical or different units of formula (IV):

RₓU′_{y}SiO_{[4-(x+y)]/2}

in which:

R, x and y correspond to the values given above,

and the monovalent radical U′ is in accordance with the following formula (V):

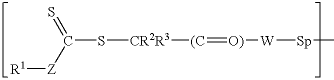

and a free-radical polymerization initiator.

The free-radical polymerization initiator may be selected from initiators conventionally used in free-radical polymerization. It may be, for example, one of the following initiators:

hydrogen peroxides, such as tert-butyl hydroperoxide, cumene hydroperoxide, t-butyl peroxy-acetate, t-butyl peroxybenzoate, t-butyl peroxyoctoate, t-butyl peroxyneodecanoate, t-butyl peroxyisobutarate, lauroyl peroxide, t-amyl peroxypivalate, t-butyl peroxypivalate, dicumyl peroxide, benzoyl peroxide, potassium persulfate, and ammonium persulfate,

azo compounds such as: 2,2′-azobis(isobutyro-nitrile), 2,2′-azobis(2-butanenitrile), 4,4′-azobis(4-pentanoic acid), 1,1′-azobis(cyclohexanecarbonitrile), 2-(t-butylazo)-2-cyanopropane, 2,2′-azobis[2-methyl-N-(1,1)-bis(hydroxymethyl)-2-hydroxyethyl]propionamide, 2,2′-azobis(2-methyl-N-hydroxyethyl]propionamide, 2,2′-azobis(N,N′-dimethyleneisobutyramidine) dichloride, 2,2′-azobis(2-amidinopropane) dichloride, 2,2′-azobis(N,N′-dimethyleneisobutyramide), 2,2′-azobis(2-methyl-N-[1,1-bis(hydroxymethyl)-2-hydroxyethyl]propionamide, 2,2′-azobis(2-methyl-N-[1,1-bis(hydroxymethyl)ethyl]propionamide), 2,2′-azobis[2-methyl-N-ethyl]propionamide, 2,2′-azobis(2-methyl-N-(2-hydroxyethyl)propionamide] and 2,2′-azobis(isobutyramide)dihydrate,

redox systems comprising combinations such as: mixtures of hydrogen peroxide, alkyl peroxide, peresters, percarbonates and the like and arbitrary iron salts, titanium salts, zinc formaldehyde sulfoxylate or sodium formaldehyde sulfoxylate, and reducing sugars; ammonium or alkali metal persulfates, perborate or perchlorate in combination with an alkali metal bisulfite, such as sodium metabisulfite, and reducing sugars; alkali metal persulfates in combination with an arylphosphinic acid, such as benzenephosphonic acid and other, similar compounds, and reducing sugars.

The amount of initiator to be used is determined such that the amount of radicals generated is not more than 20 mol % relative to the amount of silicone precursor compound (IV), preferably not more than 5 mol %.

As ethylenically unsaturated monomer use is made of those mentioned above for defining the fraction of the copolymer soluble in L2.

It should be specified, moreover, that butadiene and chloroprene correspond to the case in which a=1 in formula (I) and (III), For the preparation of the hybrid copolymers of formula (I) for which X=H and X′=NH₂ it is preferred to use, as ethylenically unsaturated monomers, vinylamine amides: for example, vinylformamide or vinylacetamide. The copolymer obtained is then hydrolyzed at acidic or basic pH.

For the preparation of the hybrid copolymers of formula (I) for which X=H and X′=OH, it is preferred to use as ethylenically unsaturated monomers, the vinyl esters of carboxylic acid, such as, for example, vinyl acetate. The copolymer obtained is then hydrolyzed at an acid or basic pH.

The types and amounts of copolymerizable monomers employed according to the present invention vary depending on the particular end application for which the hybrid copolymer is intended.

According to a first preferred version, the hybrid silicone and organic copolymer is composed of a linear silicone skeleton comprising from 1 to 300 units of formula (I), preferably from 1 to 200, and carrying from 1 to 50 radicals U, preferably from 1 to 10.

According to a second version, at least one of the monovalent radicals U′ is preferably of formula (VI):

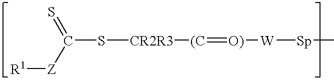

in which R² and R³, which are identical or different, represent a hydrogen atom or an optionally substituted alkyne, alkene, aryl, acyl, alkyl, alkoxycarbonyl or cyano group; and W has a definition identical to that given above, and is preferably an oxygen atom. Examples that will be mentioned include the silicone precursors in which U′ corresponds to:

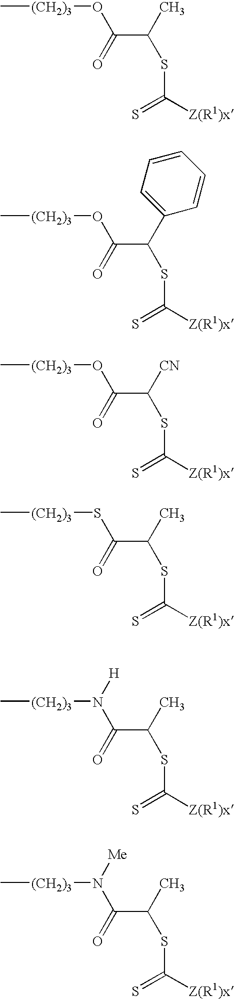

According to a third version of the invention, at least some of the monovalent radicals U′ of the silicone precursor(s) (IV) and thus at least some of the groups U of the hybrid copolymer obtained are such that Z is an oxygen atom and/or a sulfur atom.

According to a fourth variant, in addition to the use of formula (I), the hybrid silicone and organic copolymer according to the invention may comprise units RₓU_{y}F_{z}SiO_{[4-(x+y+z)]/2} (XIV) in which:

x is 0, 1, 2 or 3, y is 0, 1, 2 or 3, and z is 1, 2 or 3, where 2≦(x+y+z)≦3,

and F is a group carrying at least one functional group such as hydroxyl, alkoxy, thiol, amine, epoxy and/or polyether.

These groups F may optionally provide complementary and/or additional properties to the hybrid copolymers prepared according to the process of the invention. They may in particular be present initially within the silicone precursor of formula (IV).

In addition to the hybrid copolymers with homopolymeric organic segments, the process which has just been described makes it possible to prepare hybrid polymers carrying organic groups in blocks (i.e. multiblock polymers). To do this, the process consists in repeating the implementation of the preparation process described above, using:

monomers that are different than in the previous implementation, and

in place of the precursor silicone compound (II), the hybrid copolymer comprising block units (I) obtained from the previous implementation.

According to this process for preparing block copolymers, when it is desired to obtain copolymers having homogeneous blocks without a composition gradient, and if all the successive polymerizations are conducted in the same reactor, it is essential that all of the monomers used in one step should have been consumed before the polymerization of the following step beings, and thus before the new monomers are introduced.

As for the process for polymerizing a monoblock copolymer, this process for polymerizing block copolymers has the advantage of leading to block copolymers which have a low polydispersity index. It also allows the molecular mass of the block polymers to be controlled.

The precursor silicone compound of general formula (IV) used in the process for preparing hybrid copolymers according to the invention may be obtained by reacting:

(i) a silicone comprising units of formula (VII):

RₓU″_{y}SiO_{[4-(x+y)]/2}

in which the monovalent radical U″ is in accordance with the following formula

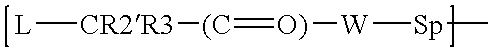

in which:

W and Sp have definitions identical with those given above,

L is a nucleophilic group, for example: Br⁻, Cl⁻, I⁻, OTs⁻, OMs⁻, (C₆H₆)—(C═O)—O⁻, (CH₃)—(C═O)—O⁻, (CF₃—(C═O)—O⁻,

(ii) with a compound selected from those of following general formulae (IX), (X) or (XI):

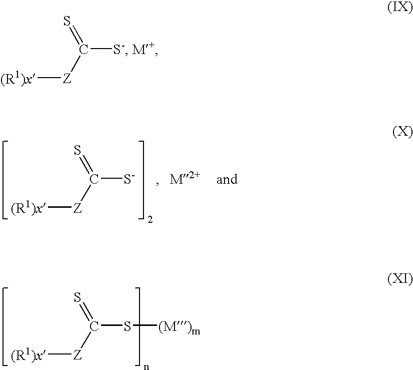

in which:

M′⁺ represents K⁺, Na⁺, NR₄⁺ or PR₄⁺, the definition of R being similar to that given for R in formula (I),

M″²⁺ represents an alkaline earth metal such as Ca²⁺, Ba²⁺ and Sr²⁺⁺,

M″′ represents Zn, Cd, m is 1 or 2, n is 1, 2, 3 or 4, and preferably m is 1 and n is 2.

This silicone of formula (VII) can be obtained in particular from (i) a silicone comprising units of formula (XII): RₓU′″_{y}SiO_{[4-(x+y)]/2}, in which the monovalent radical U′″ is of formula (XIII): —Sp—WH, and (ii) a compound of formula:

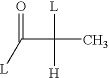

The polymerization may be conducted without solvent, in solution or in emulsion. It is preferably implemented in emulsion.

The process is preferably implemented in semicontinuous fashion.

The temperature may vary between the ambient temperature and 150° C., depending on the nature of the monomers used.

In general, during the polymerization, the instantaneous copolymer content relative to the instantaneous amount of monomer and copolymer is between 50 and 99% by weight, preferably between 75 and 99%, more preferably still between 90 and 99%. This content is maintained, in a known manner, by controlling the temperature and the rate of addition of the reactants and of the polymerization initiator.

Finally, the process is generally implemented in the absence of a UV source.

It should be noted that it may be advantageous to modify chemically the xanthate ends of the resultant copolymer, employing any method known to the skilled worker, such as, for example, a step of hydrolysis.

The process and the polymers obtained by this process are described in French application no. 00/09722, filed Jul. 25, 2000.

Other methods of synthesizing this type of copolymer may be employed, particularly that described in international applications WO 00/71606 and WO 00/71607.

According to one particular embodiment of the invention the amount of copolymer represents from 0.5 to 10% by weight of the disperse phase, preferably between 1 and 4% by weight relative to the same reference.

The disperse phase of the emulsion may further comprise at least one soluble active substance (hydrophobic active substance).

More particularly the active substance or substances are selected from active substances which can be used in the field of the treatment of the skin and/or hair. Included among these in particular, alone or in mixtures, are the following:

lipophilic vitamins, such as vitamin A and its derivatives, especially its esters, such as the acetate, palmitate, and propionate, vitamin B2, panthothenic acid, vitamin D, and vitamin E;

UV absorbers, such as the aminobenzoate derivatives of PABA and PARA type, salicylates, cinnamates, anthranilates, dibenzoylmethanes, and camphor derivatives; antiaging agents such as, in particular, retinoids, fat-soluble vitamins, vitamin C derivatives such as the esters of the acetate, propionate, and palmitate type; ceramides, pseudoceramides, phospholipids, fatty acids, fatty alcohols, cholesterol, sterols, and mixtures thereof. As preferred fatty acids and preferred alcohols mention may be made more particularly of those which possess linear or branched alkyl chains containing 12 to 20 carbon atoms. The compound involved may in particular be linoleic acid;

anticellulite agents, such as, in particular, isobutylmethylxanthine and theophylline;

antiacne agents, such as, for example, resorcinol, resorcinol acetate, benzoyl peroxide, and numerous natural compounds; bactericides;

antimicrobial agents may be selected from thymol, menthol, triclosan, 4-hexylresorcinol, phenol, eucalyptol, benzoic acid, benzoic peroxide, and butyl paraben;

aromas, perfumes, and essential oils, such as, in particular, the essential oils/essences cited above in the context of the description of L2, but also ambergris, benjamin, clove, civet, jasmine, sandalwood, vetiver, musk, myrrh, and iris, or else aldehydes and esters, such as, for example, cinnamyl acetate, cinnamaldehyde, p-methylanisole, acetaldehyde, benzaldehyde, vanillin, decanal, nerol, citral, 2,6-dimethyloctanal, and 2-ethylbutyraldehyde;

fixatives and/or styling agents for the hair, such as, for example, the vinyl acetate/crotonate/vinyl neodecanoate copolymers sold under the name Resyn® 28-2942 and Resyn® 28-2930 from National Starch.

Where the disperse phase comprises one or more hydrophobic active substances, and where the active substance or substances are not used additionally as liquid L2, their amount represents more particularly 10 to 50% by weight of said disperse phase (liquid L2).

It should be noted that the disperse phase itself may be considered as a hydrophobic active substance. Conversely, the active substance or substances may be considered as a disperse phase.

The invention additionally provides a dispersion of the emulsion comprising liquids L1 and L2, dispersed in an external aqueous phase (continuous external aqueous phase).

According to this subject of the invention, the external aqueous phase comprises at least one nonionic surfactant and/or at least one nonionic amphiphilic polymer, alone or in mixtures, optionally in combination with one or more anionic surfactants and/or one or more anionic amphiphilic polymers.

The surfactant is preferably selected from nonionic polyoxyalkylenated surfactants which are at least partly miscible in the aqueous phase.

The polyoxyalkylenated surfactant of the external aqueous phase is selected advantageously from the following surfactants, alone or in a mixture: alkoxylated fatty alcohols; alkoxylated triglycerides; alkoxylated fatty acids; alkoxylated sorbitan esters, alkoxylated fatty amines; alkoxylated di(1-phenyl-ethyl)phenols; alkoxylated tri(1-phenylethyl)phenols; alkoxylated alkylphenols; the number of alkoxylated units, more particularly oxyethylenated and/or oxypropylenated units, is such that the HLB value is greater than or equal to 10.

As regards the anionic or nonionic amphiphilic polymers, a polymer is employed which comprises at least two blocks, one of them being hydrophilic and the other hydrophobic. That which was indicated previously in the context of the description of the monomers which are hydrophilic and nonionic, ionic and those which are hydrophobic and can be used for the preparation of the comb or block copolymers.

Said amphiphilic polymers may advantageously be obtained by what is termed living or controlled free-radical polymerization. For nonlimitative examples of controlled or living polymerization processes reference may be made in particular to the applications WO 98/58974, WO 00/75207 and WO 01/42312 (xanthate), WO 98/01478 (dithio esters), WO 99/03894 (nitroxides); WO 99/31144 (dithiocarbamates), WO 02/26836 (dithiocarbazates); WO 02/10223 (dithiophosphoro-esters), WO 96/30421 (atom transfer polymerization—ATRP).

The amphiphilic polymers may also be obtained by anionic polymerization.

They may likewise be prepared by employing ring-opening polymerizations (especially anionic), or by chemical modification of the polymer.

More particularly, as regards the present amphiphilic polymer, preferably polyoxyalkylenated, of the external aqueous phase, it may be selected from polymers which are at least partly miscible in the external aqueous phase and preferably from polyethylene glycol/polypropylene glycol/polyethylene glycol triblock copolymers.

It is specified that polymers of polyvinyl alcohol type or of polyacrylic acid/polybutyl acrylate/polyacrylic acid triblock type may be used for this purpose.

The amount of anionic surfactant and/or of nonionic amphiphilic polymer is advantageously between 0.5 and 10% by weight relative to the weight of the emulsion (L2 in L1), preferably between 1 and 5% by weight of the emulsion.

Among the anionic surfactants which can be combined with at least one nonionic surfactant and/or at least one nonionic polymer, mention may be made, inter alia, of the following, alone or in mixtures:

alkyl ester sulfonates, for example of formula R—CH(SO₃M)—COOR′, in which R represents a C₈-C₂₀, preferably C₁₀-C₁₆, alkyl radical, R′ a C₁-C₆, preferably C₁-C₃, alkyl radical, and M an alkali metal cation (sodium, potassium or lithium), a substituted or unsubstituted ammonium cation (methyl-, dimethyl-, trimethyl-, tetramethyl-ammonium, dimethylpiperidinium, etc.) or a cation derived from an alkanolamine(monoethanolamine, diethanolamine, triethanolamine, etc.). Very particular mention may be made of the methyl ester sulfonates whose radical R is C₁₄-C₁₆; alkylbenzene-sulfonates, more particularly C₉-C₂₀ alkylbenzene-sulfonates, primary or secondary alkylsulfonates, especially C₈-C₂₂ alkylsulfonates, alkylglycerol sulfonates, sulfonated polycarboxylic acids, such as, for example, those described in GB 1082179, and paraffinsulfonates;

alkyl sulfates for example of formula ROSO₃M, in which R represents a C₁₀-C₂₄, preferably C₁₂-C₂₀, alkyl or hydroxyalkyl radical, M representing a hydrogen atom or a cation of the same definition as above, and their polyalkoxylated (ethoxylated (EO), propoxylated (PO), or combinations thereof) derivatives, such as, for example, sodium dodecyl sulfate;

alkyl ether sulfates for example of formula RO(CH₂CH₂O)ₙSO₃M, in which R represents a C₁₀-C₂₄, preferably C₁₂-C₂₀, alkyl or hydroxyalkyl radical, M representing a hydrogen atom or a cation of the same definition as above, n bearing generally from 1 to 4, and their polyalkoxylated (ethoxylated (EO), propoxylated (PO), or combinations thereof) derivatives, such as, for example, lauryl ether sulfate with n=2;

alkylamide sulfates, for example of formula RCONHR′OSO₃M, in which R represents a C₂-C₂₂, preferably C₆-C₂₀, alkyl radical, R′ a C₂-C₃ alkyl radical, M representing a hydrogen atom or a cation of same definition as above, and their polyalkoxylated (ethoxylated (EO), propoxylated (PO), or combinations thereof) derivatives;

salts of saturated or unsaturated fatty acids, for example such as the C₈-C₂₄, preferably C₁₄-C₂₀, compounds, N-acyl-N-alkyltaurates, alkylisethion-ates, alkylsuccinamates and alkylsulfosuccinates, sulfosuccinate monoesters or diesters, N-acyl-sarcosinates, and polyethoxycarboxylates; and

alkyl and/or alkyl ether and/or alkylaryl ether phosphate esters.

The amount of anionic surfactant, if used, is preferably between 0.5 and 5% by weight relative to the weight of the emulsion, advantageously between 0.5 and 2% by weight relative to the same reference.

More particularly, the weight ratio of emulsion relative to the external aqueous phase is between 30/70 and 90/10, preferably between 50/50 and 90/10.

According to one version of the invention the dispersion comprises at least one thickener. More particularly, said thickener is located in the continuous external aqueous phase.

The thickener is selected preferably from polysaccharides, such as xanthan gum and guar, alone or in mixtures.

Moreover, the amount of thickener is advantageously between 0.1 and 2% by weight of the dispersion (emulsion and continuous external aqueous phase).

On an ad hoc basis, it may be advantageous to incorporate at least one salt and/or sugar and/or polysaccharide into the continuous external aqueous phase. Thus, in the case in which the dispersion according to the invention (emulsion and external aqueous phase) is mixed with a multiple emulsion of water/oil/water type, it may be necessary to equilibrate the osmotic pressures of the external aqueous phase of the dispersion according to the invention (which will then be the same as the external aqueous phase of the multiple emulsion) and of the internal aqueous phase of the multiple emulsion.

Consequently, and if necessary, the continuous external aqueous phase of the dispersion according to the invention may comprise at least one salt selected from alkali metal or alkaline-earth metal halides, or alkali metal or alkaline-earth metal sulfates, or a mixture thereof, the concentration of salt in the continuous external aqueous phase being between 0.05 and 1 mol/l, preferably from 0.1 to 0.4 mol/l.

The continuous external aqueous phase may further comprise at least one sugar or at least one polysaccharide or mixtures thereof; the concentration of sugar and/or polysaccharide is such that the osmotic pressure of the continuous external aqueous phase comprising the sugar and/or polysaccharide corresponds to the osmotic pressure of a continuous external aqueous phase containing from 0.05 to 1 mol/l of salt.

The invention further provides a dispersion of the emulsion according to the invention in a continuous external organic phase.

More precisely, the emulsion according to the invention is dispersed in an external organic phase which is immiscible with the continuous phase of said emulsion.

As far as the nature of the continuous organic phase in which the emulsion according to the invention is dispersed is concerned it may be selected from the compounds listed above in the context of the description of the liquid L2 of the emulsion. This continuous external organic phase may likewise be of the same chemical nature as the disperse phase of the emulsion according to the invention, or not.

Furthermore, the continuous external organic phase comprises a stabilizer. Advantageously, this stabilizer is selected from comb or block copolymers of which one fraction is soluble in the disperse phase (in other words the continuous phase of the emulsion according to the invention) and the other in the continuous external organic phase, the fraction soluble in the continuous external organic phase being greater than the fraction soluble in the disperse phase.

Everything described above in relation to the copolymer employed to stabilize the emulsion according to the invention (L2/L1) will also apply in the case of the stabilizer stabilizing the emulsion dispersed in a continuous external organic phase, subject to the condition that the greater fraction of the polymer is that which is soluble in the continuous external organic phase, being different in nature from that which forms the continuous phase of the emulsion according to the invention.

Finally, the weight ratio of emulsion (L2/L1) relative to the continuous external organic phase is, in accordance with one version, between 30/70 and 90/10, preferably between 50/50 and 90/10.

One method of obtaining the emulsion according to the invention will now be described, and consists in preparing, on the one hand, a first mixture comprising the disperse phase, optionally comprising the active substance, and, on the other hand, a second mixture comprising the continuous phase and the polymer, and then in mixing the first mixture with the second, with stirring.

Where the continuous phase is of low viscosity (viscosity less than 1 Pa.s), stirring is preferably vigorous, and may advantageously be effected by the use of an apparatus of the Ultra-Turrax® or Microfluidizer type, or any high-pressure homogenizer.

Where the continuous phase is viscous (viscosity greater than or equal to 1 Pa.s, preferably greater than or equal to 5 Pa.s), agitation may advantageously be effected using a gate paddle.

The emulsion is generally prepared at a temperature of between 20 and 80° C.

The stirring time may be determined readily by the skilled worker, and depends on the type of apparatus employed. It is preferably sufficient to give an average droplet size of between 0.1 and 10 μm, preferably between 0.1 and 5 μm, (measured by means of a Horiba granulometer).

As far as a method of preparing the dispersion of the emulsion in a continuous external aqueous phase is concerned, a mixture is prepared of the continuous external aqueous phase comprising the surfactant and/or the nonionic amphiphilic polymer and, optionally, the anionic surfactant, and the emulsion according to the invention is added to this mixture, and then, optionally, an aqueous thickener solution is added to the emulsion thus obtained.

According to the version in which the dispersion of the emulsion in the continuous external aqueous phase is mixed with a multiple water/oil/water emulsion, adjustment is made, preferably, to the osmotic pressures of the continuous external aqueous phase and of the internal aqueous phase of the multiple emulsion. This is done in particular by introducing the salt and/or sugar and/or polysaccharide, in the form of an aqueous solution, into the dispersion according to the invention, before or after the thickener has been added to the dispersion.

As regards the preparation of the dispersion of the emulsion in a continuous external continuous organic phase, a mixture of the organic phase comprising the stabilizer is prepared, and the emulsion according to the invention is added thereto.

The dispersed dispersion, in whatever version, is prepared preferably with stirring, with the emulsion being added slowly at the start.

Stirring may be carried out by means of a gate paddle. Typically, the stirring speed is relatively slow, of the order of 400 revs/minute.

The emulsion according to the invention, the dispersion of the emulsion in a continuous external aqueous phase, and the dispersion of the emulsion in a continuous external organic phase may be used in the field in the treatment of the skin and/or hair, more particularly as additives for such formulations.

The amount of emulsion or dispersion is preferably such that the amount of active substance(s) in the cosmetic or dermatological formulation is between 0.01 and 50% by weight of said formulation.

The cosmetic compositions provided by the invention may be formulated as a large number of types of products for the skin, hair, eyelashes and/or nails, shampoos for hair or for the body, body or facial cleansing gels, liquid soaps, foaming compositions for the bath, conditioners, formulations for styling hair and for making it easier to comb hair, hand and body lotions, products regulating moisturization of the skin, toilet milks, makeup remover compositions, depilatory products, creams or lotions for protecting against the sun and ultraviolet radiation, beauty creams, antiacne preparations, makeup formulations of mascara, foundation or nail varnish type, products intended for application to the lips, etc.

Cosmetic formulations, in addition to the emulsion or dispersion according to the invention, comprise additives conventional in the art.

Accordingly, the formulations may comprise surfactants, whose amount may vary between approximately 3-40% by weight relative to the formulation.

Among suitable surfactants mention may be made, by way of anionic surfactants, of the following: alkyl ester sulfonates, alkylbenzenesulfonates, primary or secondary alkylsulfonates, alkylglycerol sulfonates, sulfonated polycarboxylic acids, and paraffinsulfonates; alkyl sulfates; alkyl ether sulfates; alkylamidesulfates; salts of saturated or unsaturated fatty acids, N-acyl-N-alkyltaurates, alkylisethionates, alkylsuccinamates and alkylsulfosuccinates, sulfosuccinate monoesters or diesters, N-acylsarcosinates, and polyethoxycarboxylates; and alkyl and/or alkyl ether and/or alkylaryl ether phosphate esters; alone or in a mixture.

Among the nonionic surfactants which can be used in the formulations, those which are suitable include the following: alkoxylated fatty alcohols; alkoxylated triglycerides; alkoxylated fatty acids; alkoxylated sorbitan esters; alkoxylated fatty amines; alkoxylated di(1-phenylethyl)phenols; alkoxylated tri(1-phenylethyl)phenols; alkoxylated alkylphenols; products resulting from the condensation of ethylene oxide with a hydrophobic compound resulting from the condensation of propylene oxide with propylene glycol, such as the Pluronic products sold by BASF; products resulting from the condensation of ethylene oxide the compound resulting from the condensation of propylene oxide with ethylenediamine, such as the Tetronic products sold by BASF; alkylpolyglycosides such as those described in U.S. Pat. No. 4,565,647; and amides of fatty acids, for example C₈-C₂₀ acids; alone or in a mixture.

The formulations may likewise comprise cationic surfactants such as alkyldimethylammonium halides, or else amphoteric or zwitterionic surfactants, such as betaines, for example lauryl betaine (Mirataine BB from the company Rhodia Chimie); sulfobetaines; amidoalkylbetaines, such as cocamidopropylbetaine (Mirataine BDJ from the company Rhodia Chimie); sultaines such as cocamidopropylhydroxysultaine (Mirataine CBS from the company Rhodia Chimie); alkylamphoacetates and alkylamphodiacetates, such as those, for example, comprising a coco chain, lauryl (Miranol C2M, C32 in particular from the company Rhodia Chimie); alkylamphopropionates or alkylamphodi-propionates (Miranol C2M SF); and alkylamphohydroxy-propylsultaines (Miranol CS).

It is possible thus to incorporate into the cosmetic composition, in dispersion or solution form, bactericides or fungicides, for the purpose of improving skin disinfection, such as triclosan, for example; antidandruff agents, such as, in particular, zinc pyrithione or octopirox; and insecticides, such as natural or synthetic pyrethroids.

The cosmetic compositions may also include agents for protecting the skin and/or hair against the aggressive effects of the sun and of UV rays. Accordingly the compositions may comprise solar filters, which are chemical compounds with high absorption for UV radiation, such as the compounds authorized in European Directive 76/768/EEC, its annexes and the subsequent amendments to said directive.

The cosmetic compositions may also include fixative resins.

These fixative resins, when present, are generally present at concentrations of between 0.01 and 10%, preferably between 0.5 and 5%.

The fixative resins forming part of the cosmetic compositions are more particularly selected from the following resins:

methyl acrylate/acrylamide copolymers, polyvinyl methyl ether/maleic anhydride copolymers, vinyl acetate/crotonic acid copolymers, octylacrylamide/methyl acrylate/butylaminoethyl methacrylate copolymers, polyvinylpyrrolidones, polyvinylpyrrolidone/methyl methacrylate copolymers, polyvinylpyrrolidone/vinyl acetate copolymers, polyvinyl alcohols, polyvinyl alcohol/crotonic acid copolymers, polyvinyl alcohol/maleic anhydride copolymers, hydroxypropylcelluloses, hydroxypropylguars, sodium polystyrenesulfonates, polyvinylpyrrolidone/ethyl methacrylate/methacrylic acid terpolymers, poly(methyl vinyl ether/maleic acid) monomethyl ethers, and polyvinyl acetates grafted onto polyoxyethylene backbones (EP 219048);

copolyesters derived from terephthalic and/or isophthalic and/or sulfoisophthalic acid, anhydride or a terephthalic and/or isophthalic and/or sulfoisophthalic diester, and a diol, such as:

polyester copolymers based on ethylene terephthalate and/or propylene terephthalate and polyoxyethylene terephthalate units (U.S. Pat. No. 3,959,230, U.S. Pat. No. 3,893,929, U.S. Pat. No. 4,116,896, U.S. Pat. No. 4,702,857, U.S. Pat. No. 4,770,666);

sulfonated polyester oligomers obtained by sulfonating an oligomer derived from ethoxylated allyl alcohol, dimethyl terephthalate and 1,2-propylenediol (U.S. Pat. No. 4,968,451);

polyester copolymers derived from dimethylterephthalate, isophthalic acid, dimethyl sulfoisophthalate, and ethylene glycol (EP 540374);

copolymers comprising polyester units derived from dimethyl terephthalate, isophthalic acid, dimethyl sulfoisophthalates, and ethylene glycol and polyorganosiloxane units (FR 2728915);

sulfonated polyester oligomers obtained by condensing isophthalic acid, dimethyl sulfosuccinate, and diethylene glycol (FR 2236926);

polyester copolymers based on propylene terephthalate and polyoxyethylene terephthalate units and terminated by ethyl units, methyl units (U.S. Pat. No. 4,711,730), or polyester oligomers terminated by alkylpolyethoxy groups (U.S. Pat. No. 4,702,857) or anionic sulfopolyethoxy groups (U.S. Pat. No. 4,721,580) or sulfoaroyl groups (U.S. Pat. No. 4,877,896);

polyester-polyurethanes, more particularly those obtained by reacting a polyester obtained from adipic acid and/or terephthalic acid and/or sulfoisophthalic acid and a diol with a prepolymer containing terminal isocyanate groups, obtained from a polyoxyethylene glycol and a diisocyanate (FR 2334698);

ethoxylated monoamines or polyamines, and polymers of ethoxylated amines (U.S. Pat. No. 4,597,898, EP 11984).

Preferentially the fixative resins are selected from polyvinylpyrrolidone (PVP), copolymers of polyvinylpyrrolidone and methyl methacrylate, the copolymer of polyvinylpyrrolidone and vinyl acetate (VA), polyethylene glycol terephthalate/polyethylene glycol copolymers, polyethylene glycol terephthalate/polyethylene glycol/polysodium sulfonate isophthalate copolymers, and mixtures thereof.

These fixative resins are preferably dispersed or dissolved in the selected vehicle.

The cosmetic compositions may also include polymeric derivatives which exert a protective function.

These polymeric derivatives may be present in amounts of the order of 0.01-10%, preferably approximately 0.1-5%, and more particularly of the order of 0.2-3% by weight.

These agents may in particular be selected from:

nonionic cellulose derivatives such as cellulose hydroxyethers, methylcellulose, ethylcellulose, hydroxypropylmethylcellulose, and hydroxybutylmethyl-cellulose;

polyvinyl esters grafted onto polyalkylenated backbones, such as the polyvinyl acetates grafted onto polyoxyethylene backbones (EP 219048);

polyvinyl alcohols.

The cosmetic compositions may also comprise plasticizers.

Said plasticizers, when present, represent between 0.1 to 20% of the formulation, preferably from 1 to 15%.

Among particularly useful plasticizers mention may be made of adipates, phthalates, isophthalates, azelates, stearates, silicone copolyols, glycols, castor oil, or mixtures thereof.

It is also possible with advantage to add metal sequestrants to these compositions, more particularly those which sequester calcium, such as citrate ions.

It is likewise possible to incorporate humectants into the cosmetic compositions; humectants include, inter alia, glycerol, sorbitol, urea, collagen, gelatin, aloe vera, hyaluronic acid or volatile water-soluble solvents such as ethanol or propylene glycol, the amounts of which may reach up to 60% by weight of the composition.

In order further to reduce the irritation or aggression of the scalp it is also possible to add water-soluble or water-dispersible polymers such as collagen or certain non-allergenic derivatives of animal or plant proteins (wheat protein hydrolysates, for example), natural hydrocolloids (guar gum, carob gum, tara gum, etc.) or hydrocolloids obtained from fermentation processes, and derivatives of these polycarbohydrates, such as nonionic modified celluloses, hydroxyethylcellulose for example, or anionic modified celluloses, such as carboxymethyl-cellulose; guar derivatives or carob derivatives, such as their nonionic derivatives (hydroxypropylguar, for example) or the anionic derivatives (carboxymethylguar and carboxymethylhydroxypropylguar).

To these compounds it is possible to add, in combination, mineral particles or powders, such as calcium carbonate, sodium bicarbonate, calcium dihydrogenphosphate, mineral oxides in powder form or in colloidal form (particles with a size smaller than or of the order of one micrometer, sometimes several tens of nanometers), such as titanium dioxide, silica, aluminum salts, which are used generally as antipers-pirants, kaolin, talc, clays and their derivatives, etc.

Preservatives, such as the methyl, ethyl, propyl and butyl esters of p-hydroxybenzoic acid, sodium benzoate, Germaben® or any chemical preventing the proliferation of bacteria or molds which is traditionally used in cosmetic compositions may also be introduced into the aqueous cosmetic compositions according to the invention, generally at a level of from 0.01 to 3% by weight.

The amount of these products is commonly adjusted in order to prevent any proliferation of bacteria, molds or yeasts in the cosmetic compositions.

Alternatively to these chemicals it is sometimes possible to use agents which modify the activity of water and which greatly increase the osmotic pressure such as carbohydrates or salts.

In order to protect the skin and/or hair from attack by the sun and UV rays it is possible to add to these formulations mineral particles such as zinc oxide, titanium dioxide or cerium oxides, in powder form or in the form of colloidal particles, alone or in a mixture. These powders may optionally be surface-treated in order to enhance the effectiveness of their anti-UV activity or in order to facilitate their incorporation into the cosmetic formulations or in order to inhibit the surface photoreactivity.

To these ingredients it is possible to add, if necessary, for the purpose of enhancing comfort when the composition is used by the consumer, one or more perfumes, colorants, among which mention may be made of the products described in annex IV (“List of coloring agents allowed for use in cosmetic products”) of European Directive 76/768/EEC of 27 Jul. 1976, known as the Cosmetics Directive, and/or opacifiers such as pigments.

Although not mandatory, the composition may also include viscosity-modifying or gelling polymers, in order to adjust the texture of the composition, such as crosslinked polyacrylates (Carbopol products, sold by Goodrich), noncationic cellulose derivatives such as hydroxypropylcellulose, carboxymethylcellulose, guars and their nonionic derivatives, xanthan gum and its derivatives, which are used alone or in combination, or the same compounds, generally in the form of water-soluble polymers modified with hydrophobic groups linked covalently to the polymer framework, as described in international application WO 92/16187, and/or water, in order to take the total of the constituents of the formulation to 100%.

The cosmetic compositions may likewise include polymeric dispersants in an amount of the order of 0.1-7% by weight, in order to control the hardness in terms of calcium and magnesium; the dispersants are such as:

water-soluble salts of polycarboxylic acids with a weight-average molecular mass of the order to 2 000 to 100 000 g/mol, obtained by polymerization or copolymerization of ethylenically unsaturated carboxylic acids such as acrylic acid, maleic acid or anhydride, fumaric acid, itaconic acid, aconitic acid, mesaconic acid, citraconic acid, methylenemalonic acid, and very particularly polyacrylates with a weight-average molecular mass of the order of 2 000 to 10 000 g/mol (U.S. Pat. No. 3,308,067), and copolymers of acrylic acid and maleic anhydride with a weight-average molecular mass of the order of 5 000 to 75 000 g/mol (EP 66915);

polyethylene glycols, for example with a weight-average molecular mass of the order of 1 000 to 50 000 g/mol.

Specific but nonlimitative examples of the invention will now be described.

### EXAMPLE 1

The object of this example is the synthesis of a polybutyl acrylate 2500—polydimethylsiloxane 14000—polybutyl acrylate 2500 triblock copolymer.

### 1a. Preparation of the Alpha, Amega-bis (xanthate) PDMS Oil

100 g of alpha,omega-carbinol silicone oil containing 193 dimethylsiloxane units (¹H NMR analysis) is dissolved in 500 ml of cyclohexane in the presence of 4 molar equivalents of triethylamine, NEt₃.

2.4 equivalents of 2-bromopropionyl bromide in solution in 100 ml of cyclohexane are introduced via a cannula.

A white precipitate is formed immediately, with a slight increase in the temperature of the reaction mixture.

The aqueous phase is subsequently separated from the organic phase. The cyclohexane is then evaporated.

The crude product obtained is subsequently filtered over a bed of silica. Evaporation of the solvents gives 102 g (84% yield) of acylation product.

### 1b. Synthesis of Polybutyl Acrylate 2500—Polydimethylsiloxane 14000—Polybutyl Acrylate 2500 Triblock Copolymer

0.0352 g of azobisisobutyronitrile are introduced into a glass reactor simultaneously with 4 g of the PDMS oil described in example 1a, 1.342 g of butyl acrylate and 12.548 g of cyclohexane.

The mixture is heated at 70° C. for 7 hours.

The solvent is then evaporated.

The following results are obtained: Mₙ=13 800 g/mol M_{w}/Mₙ=1.98

### EXAMPLE 2

The object of this example is to obtain a 50/50 silicone oil (L1)/vegetable oil (L2) emulsion.

A mixture is prepared comprising the copolymer obtained at the end of the preceding example in a silicone oil (Mirasil DM 300, sold by Rhodia Chimie).

The respective amounts of these compounds are such that the final silicone oil (L1)/vegetable oil (L2) emulsion contains 2.5% by weight of copolymer and 45.5% of silicone; these percentages are expressed relative to the total weight of the emulsion L2/L1.

The mixture is obtained by stirring using a gate paddle.

A solution is obtained.

The vegetable oil (sunflower oil, sold by the company Bertin, Lagny-le-Sec, France) is then poured dropwise onto the solution obtained beforehand, with gate paddle stirring of the order of 600 revs/minute. Stirring is then continued using an Ultra-turrax (9500 revolutions/minute approximately) for three minutes.

This gives a stable emulsion.

### EXAMPLE 3

The object of this example is to obtain a dispersion of the emulsion thus obtained in an aqueous phase (emulsion content 80% by weight/aqueous phase content 20% by weight).

A 10% strength by weight aqueous solution of Arlatone F127G (*) (ICI—Uniquema) is prepared.

Subsequently, the emulsion obtained according to example 2 if added, slowly and continuously, to said solution, with gate paddle stirring at 300 revs/minute, over approximately three to five minutes.

The concentrated dispersion thus obtained is stable.

(*) Arlatone F127G: HO(CH₂CH₂O)ₓ(OCH(CH₃)CH₂O)_{y}(CH₂CH₂O)_{z}H, meeting the following inequation: 82<x+z<90 and the polymer contains 7 PO units per mole of product).

### EXAMPLE 4

The object of this example is to obtain a dilute dispersion of the emulsion thus obtained in an aqueous phase (emulsion content 50% by weight/aqueous phase content 50% by weight).

A 2% strength by weight solution of Rhodopol 23 (xanthan gum; Rhodia Chimie) is prepared.

This solution is then added by spatula to the dispersion obtained in example 3 to give a final Rhodopol 23 concentration of 0.5% relative to the total weight of the final dispersion.

The dispersion thus obtained is stable.

### EXAMPLE 5

The object of this example is to obtain an ethanol/silicone oil/water emulsion.

### Preparation of the Ethanol/Silicone Oil (80/20) Emulsion

The copolymer employed is a polybutyl acrylate 3000—polydimethylsiloxane 35000—polybutyl acrylate 3000 triblock copolymer. It is obtained in accordance with the method described in example 1 except that the alpha,omega-carbinol silicone oil contains 422 dimethylsiloxane units (¹H NMR analysis) and that the amounts of butyl acrylate used are adapted so as to give polybutyl acrylate 3000 blocks.

First of all, 1 g of the aforementioned copolymer is added to 1 g of silicone oil (Rhodorsil 47V1000; sold by Rhodia Chimie). The mixture is then brought to 5° C. and homogenized at 400 rpm using a solid gate paddle.

8 g of a resin solution in ethanol (Resyn 28-2930, sold by National Starch; 40% resin solution neutralized to 90% by adding 95% 2-amino-2-methyl-1-propanol, Aldrich) are added dropwise to the silicone phase. At the end of addition, shearing is maintained at 400 rpm for 20 minutes, for refining.

A stable emulsion is obtained.

### Preparation of the [Ethanol/Silicone Oil]/Water (91/9) Dispersion

The external aqueous phase is composed of a 10% strength solution of Arlatone F127G in water.

0.4 g of 10% strength Arlatone F127G solution is introduced into 4 g of the emulsion obtained above.

The mixture is homogenized (Eurostar IKA homogenizer, Labortechnik, Junke & Junkel; equipped with a solid gate paddle, speed increased to 400 rpm). When the mixture appears homogeneous, stirring for 5 minutes approximately.

A stable emulsion is obtained.

1-32. (Canceled)

33. An emulsion comprising two nonaqueous liquids, L1 and L2, which are mutually immiscible, the liquid L1, which represents the continuous phase of the emulsion or the disperse phase of the emulsion, is a silicone, the emulsion being stabilized by at least one comb or block copolymer of which one fraction is soluble in the disperse phase and the other in the continuous phase, the fraction soluble in the continuous phase being greater than the fraction soluble in the disperse phase.

34. The emulsion according to claim 33, wherein the liquid L1 is selected from silicones, alone or in a mixture, which are composed in whole or in part of units of formula:
R′₃₋ₐRₐSiO_{1/2} (unit M) and/or R₂SiO (unit D) in which formulae:
a is an integer from 0 to 3;
the radicals R are identical or different and represent:
a saturated or unsaturated aliphatic hydrocarbon group containing 1 to 10 carbon atoms;
an aromatic hydrocarbon group containing 6 to 13 carbon atoms;
a polar organic group bonded to the silicon by an Si—C or Si—O—C bond;
the radicals R′ are identical or different and represent:
a saturated or unsaturated aliphatic hydrocarbon group containing 1 to 10 carbon atoms;
an aromatic hydrocarbon group containing 6 to 13 carbon atoms;
an —OH function;
an amino- or amido-functional group containing 1 to 6 carbon atoms, bonded to the silicon by an Si—N bond.

35. The emulsion according to claim 33, wherein the liquid L2 is selected from the group consisting of:
organic waxes, fats, oils of animal or plant origin;
mineral waxes/oils;
products obtained from the alcoholysis of the aforementioned oils;
essential oils;
mono-, di- and tri-glycerides;
saturated or unsaturated fatty acids containing 10 to 40 carbon atoms; the esters of such acids with alcohol containing 1 to 6 carbon atoms;
saturated or unsaturated monoalcohols containing 2 to 40 carbon atoms;
polyols containing 2 and 10 carbon atoms; and silicone oils.

36. The emulsion according to claim 33, wherein the liquid L1 represents the continuous phase of the emulsion.

37. The emulsion according to claim 33, wherein the proportion by weight of liquid L2 relative to liquid L1 is between 10/90 and 90/10.

38. The emulsion according to claim 33, wherein the fraction soluble in L1 of the comb or block copolymer is derived from the silicones composing L1, said silicones optionally carrying reactive functions.

39. The emulsion according to claim 33, wherein the fraction soluble in L2 of the comb or block copolymer derives from the polymerization of at least one monomer selected from the group consisting of:
esters of linear, branched, cyclic or aromatic monocarboxylic or polycarboxylic acids containing at least one ethylenic unsaturation,
α,β-ethylenically unsaturated nitrites, vinyl ethers, vinyl esters, vinylaromatic monomers, and vinyl halides or vinylidene halides, and
linear or branched, aromatic or nonaromatic hydrocarbon monomers containing at least one ethylenic unsaturation.

40. The emulsion according to claim 33, wherein the copolymer is a copolymer comprising at least three blocks.

41. The emulsion according to claim 33, wherein the copolymer represents an amount of from 0.5 to 10% by weight of the disperse phase.

42. The emulsion according to claim 33, wherein the disperse phase further comprises at least one active substance soluble in said disperse phase.

43. The emulsion according to claim 42, wherein the active substance is selected from the group consisting of:
lipophilic vitamins;
UV absorbers; antiaging agents;
anticellulite agents;
antiacne agents;
antimicrobial agents; bactericides;
aromas; essential oils; perfumes;
styling agents;
and fixatives for the hair.

44. The emulsion according to claim 43, wherein the active substance is hydrophobic and represents 10 to 50% by weight of the disperse phase.

45. A dispersion comprising the emulsion as defined in claim 33, dispersed in a continuous external aqueous phase comprising at least one nonionic surfactant or at least one nonionic amphiphilic polymer, optionally in combination with one or more anionic surfactants or anionic amphiphilic polymers.

46. The dispersion according to claim 45, wherein the nonionic is optionally polyoxyalkylenated, and at least partly miscible in the external aqueous phase is selected from the group consisting of:
alkoxylated fatty alcohols,
alkoxylated triglycerides,
alkoxylated fatty acids,
alkoxylated sorbitan esters,
alkoxylated fatty amines,
alkoxylated di(1-phenylethyl)phenols,
alkoxylated tri(1-phenylethyl)phenols, and
alkoxylated alkylphenols, and wherein the number of alkoxylated units, is such that the HLB value is greater than or equal to 10.

47. The dispersion according to claim 45, wherein the amphiphilic polymer of the external aqueous phase is at least partly miscible in the external aqueous phase and is a polyethylene glycol/polypropylene glycol/polyethylene glycol triblock copolymer.

48. The dispersion according to claim 45, wherein the nonionic surfactant or the nonionic amphiphilic polymer is present in an amount of between 0.5 and 10% by weight relative to the weight of the emulsion.

49. The dispersion according to claim 45, comprising said anionic surfactant selected from the group consisting of:
alkyl ester sulfonates, alkylbenzenesulfonates, primary or secondary
alkylsulfonates, alkylglycerol sulfonates, sulfonated polycarboxylic acids,
paraffinsulfonates;
alkyl sulfates;
alkyl ether sulfates;
alkylamide sulfates;
salts of saturated or unsaturated fatty acids, N-acyl-N-alkyltaurates,
alkylisethionates, alkylsuccinamates and alkylsulfosuccinates, sulfosuccinate
monoesters, sulfosuccinate diesters, N-acylsarcosinates,
polyethoxycarboxylates; and
alkyl ether phosphate esters, alkyl ether ether phosphate esters, alkylaryl ether phosphate esters.

50. The dispersion according to claim 49, wherein the anionic surfactant is present in an amount of between 0.5 and 5% by weight relative to the weight of the emulsion.

51. The dispersion according to claim 45, wherein the emulsion presents a weight ratio relative to the continuous external aqueous phase of between 30/70 and 90/10.

52. The dispersion according to claim 45, further comprising at least one thickener.

53. The dispersion according to claim 52, wherein the thickener is selected from polysaccharides.

54. The dispersion according to claim 52, wherein the thickener is present in an amount of between 0.1 and 2% by weight of the emulsion and of the continuous external aqueous phase.

55. The dispersion according to claim 45, wherein the continuous external aqueous phase further comprises at least one salt selected from alkali metal or alkaline-earth metal halides, or alkali metal or alkaline-earth metal sulfates, with a salt concentration in the continuous external aqueous phase of between 0.05 and 1 mol/l.

56. The dispersion according to claim 45, wherein the continuous external aqueous phase further comprises at least one sugar, or at least one polysaccharide, with a sugar or polysaccharide concentration being such that the continuous external aqueous phase comprising the sugar or polysaccharide present an osmotic pressure corresponding to the osmotic pressure of an internal aqueous phase containing from 0.05 to 1 mol/l of salt.

57. The dispersion comprising the emulsion as defined in claim 33, dispersed in a continuous external organic phase comprising a stabilizer comprising at least one comb or block copolymer, one fraction of which is soluble in the disperse phase and the other in the continuous external organic phase, the fraction soluble in the continuous external organic phase being greater than the fraction soluble in the disperse phase.

58. The dispersion according to claim 57, wherein the continuous external organic phase is immiscible with the continuous phase of the emulsion.

59. The dispersion according to claim 57, wherein the emulsion has a weight ratio relative to the continuous external organic phase of between 30/70 and 90/10.

60. A method for the preparation of an emulsion as defined in claim 33, comprising the steps of:
a) preparing a first mixture comprising the disperse phase optionally comprising the active substance, and
b) preparing a second mixture comprising the continuous phase and the polymer, and, then,
c) adding with stirring the first mixture obtained in step a) to the second mixture obtained in step b) in order to obtain said emulsion.

61. A formulation for the treatment of skin or hair comprising an emulsion as defined in claim 33.

62. A formulation for the treatment of skin or hair comprising a dispersion as defined in claim 45.
